(19) United States
(12) Patent Application Publication
(10) Pub. No.: US 2017/0081258 A1
(43) Pub. Date: Mar. 23, 2017

(54) PROCESS FOR THE AROMATIZATION OF A METHANE-CONTAINING GAS STREAM USING TITANIUM ALLOY HYDROGEN ACCEPTOR PARTICLES

(71) Applicant: SHELL OIL COMPANY, Houston, TX (US)

(72) Inventors: Steven Sangyun LIM, Kingwood, TX (US); Peter Tanev TANEV, Katy, TX (US); David Morris HAMILTON, JR., Sugar Land, TX (US); Suchismita SANYAL, Bengaluru (IN)

(21) Appl. No.: 15/183,001

(22) Filed: Jun. 15, 2016

Related U.S. Application Data
(60) Provisional application No. 62/220,285, filed on Sep. 18, 2015.

(51) Int. Cl. C07C 2/76

(52) U.S. Cl. CPC C07C 2/76; C07C 2529/48

(57) ABSTRACT

Implementations of the disclosed subject matter provide a process for the aromatization of a methane-containing gas stream that includes contacting the methane-containing gas stream in a reaction zone of an aromatization reactor comprising an aromatization catalyst and a titanium alloy hydrogen acceptor under methane-containing gas aromatization conditions to produce a product stream comprising aromatics and hydrogen, wherein at least a portion of the produced hydrogen is bound by the titanium alloy hydrogen acceptor in the reaction zone and removed from the product and the reaction zone as titanium hydride, and wherein the titanium alloy hydrogen acceptor is a single phase alloy.

### CROSS-REFERENCE TO RELATED APPLICATIONS

This application claims priority to U.S. Provisional Application Ser. No. 62/220,285 filed Sep. 18, 2015, the entire disclosure of which is hereby incorporated by reference. This application is also related to co-pending U.S. patent application Ser. No. 14/395,819, entitled “AROMATIZATION OF A METHANE-CONTAINING GAS STREAM”, which claims priority to U.S. Provisional Application No. 61/636,915 filed on Apr. 23, 2012, the disclosure of which is incorporated herein by reference. This application is also related to co-pending U.S. patent application Ser. No. 14/395,821, entitled “A PROCESS FOR THE AROMATIZATION OF A METHANE-CONTAINING GAS STREAM”, which claims priority to U.S. Provisional Application No. 61/636,906 filed on Apr. 23, 2012, the disclosure of which is incorporated herein by reference.

### TECHNICAL FIELD OF THE INVENTION

This invention relates to a process for the aromatization of a methane-containing gas stream in a reactor containing both catalyst and titanium alloy hydrogen acceptor particles, wherein the titanium alloy hydrogen acceptor particles bind the produced hydrogen insitu from the methane aromatization reaction thereby shifting the thermodynamic equilibrium of the reaction and resulting in a significantly higher CH4 conversion and aromatics yields than the maximum allowable by the equilibrium.

### BACKGROUND

The aromatic hydrocarbons (specifically benzene, toluene and xylenes) are the main high-octane bearing components of the gasoline pool and important petrochemical building blocks used to produce high value chemicals and a variety of consumer products, for example, styrene, phenol, polymers, plastics, medicines, and others. Since the late 1930's, aromatics are primarily produced by upgrading of oil-derived feedstocks via catalytic reforming or cracking of heavy naphthas. However, occasional severe oil shortages and oil price spikes result in severe aromatics shortages and aromatics price spikes. Therefore, there is a need to develop new, independent from oil, commercial routes to produce high value aromatics from highly abundant and inexpensive hydrocarbon feedstocks such as methane or stranded natural gas (which typically contains about 80-90% vol. methane).

There are enormous proven reserves of stranded natural gas around the world. According to some estimates, the world reserves of natural gas are at least equal to those of oil. However, unlike the oil reserves that are primarily concentrated in a few oil-rich countries and are extensively utilized, upgraded and monetized, the natural gas reserves are much more broadly distributed around the world and significantly underutilized. Many developing countries that have significant natural gas reserves lack the proper infrastructure to exploit them and convert or upgrade them to higher value products. Quite often, in such situations, natural gas is flared to the atmosphere and wasted. Because of the above reasons, there is enormous economic incentive to develop new technologies that can efficiently convert methane or natural gas to higher value chemical products, specifically aromatics.

In 1993, Wang et al., (Catal. Lett. 1993, 21, 35-41), discovered a direct, non-oxidative route to partially convert methane to benzene by contacting methane with a catalyst containing 2.0% wt. Molybdenum on an H-ZSM-5 zeolite support at atmospheric pressure and a temperature of 700° C. Since Wang's discovery, numerous academic and industrial research groups have become active in this area and have contributed to further developing various aspects of the direct, non-oxidative methane to benzene catalyst and process technology. Many catalyst formulations have been prepared and tested and various reactor and process conditions and schemes have been explored.

Despite these efforts, a direct, non-oxidative methane aromatization catalyst and process cannot yet be commercialized. Some important challenges that need to be overcome to commercialize this process include: (i) the very low, as dictated by thermodynamic equilibrium, per pass conversion and benzene yield (for example, 10% wt. and 6% wt., respectively at 700° C.); (ii) the fact that the reaction is favored by high temperature and low pressure; (iii) the need to separate the produced aromatics and hydrogen from unreacted (mainly methane) hydrocarbon off gas and (iv) the rapid coke formation and deposition on the catalyst surface and corresponding relatively fast catalyst deactivation. Among these challenges, overcoming the thermodynamic equilibrium limitations and significantly improving (e.g., by greater than 3 times) the conversion and benzene yield per pass has the potential to enable the commercialization of an efficient, direct, non-oxidative methane-containing gas aromatization process.

The methane aromatization reaction can be described for the particular case of methane to benzene as follows:

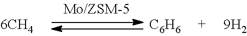

According to the reaction, 6 molecules of methane are required to generate a molecule of benzene. It is also apparent that, the production of a molecule of benzene is accompanied by the production of 9 molecules of hydrogen. Simple thermodynamic calculations revealed and experimental data have confirmed that, the methane aromatization at atmospheric pressure is equilibrium limited to about 10 or 20% wt. at reaction temperatures of 700° C. or 800° C., respectively. In addition, experimental data showed that the above conversion levels correspond to about 6 and 11.5% wt. benzene yield at 700° C. and 800° C., respectively. The aforementioned low per pass methane conversions and benzene yields are not attractive enough to provide an economic justification for scale-up and commercialization of a methane containing gas aromatization process.

Therefore, there is a need to develop an improved direct, non-oxidative methane aromatization process that provides for significantly higher (than those allowed by the thermodynamic equilibrium) methane conversion and benzene yields per pass by implementing an insitu hydrogen removal from the reaction zone.

### BRIEF SUMMARY

The invention provides a process for the aromatization of a methane-containing gas stream comprising: contacting the methane-containing gas stream in a reaction zone of a reactor comprising an aromatization catalyst and a titanium alloy hydrogen acceptor under methane-containing gas aromatization conditions to produce a product stream comprising aromatics and hydrogen wherein at least a portion of the produced hydrogen is bound by the titanium alloy hydrogen acceptor in the reaction zone and removed from the product stream and the reaction zone, and wherein the titanium alloy hydrogen acceptor is a single phase alloy.

The invention further provides a novel process and reactor schemes that employ single or multiple catalysts and/or titanium alloy hydrogen acceptor beds.

The invention also provides several catalyst and/or titanium alloy hydrogen acceptor recycle and regeneration process schemes. According to these schemes, the catalyst and/or titanium alloy hydrogen acceptor particles are regenerated simultaneously or separately in single or in separate vessels and then returned to the reactor for continuous (uninterrupted) production of aromatics and hydrogen. The aforementioned insitu hydrogen removal in the reaction zone allows for overcoming the thermodynamic equilibrium limitations of the methane aromatization reaction and for shifting the reaction equilibrium to the right. This results in significantly higher and economically more attractive methane-containing gas stream conversion and benzene yields per pass relative to the case without hydrogen removal, i.e. without titanium alloy hydrogen acceptor in the reaction zone. Additional features, advantages, and embodiments of the disclosed subject matter may be set forth or apparent from consideration of the following detailed description, drawings, and claims. Moreover, it is to be understood that both the foregoing summary and the following detailed description are examples and are intended to provide further explanation without limiting the scope of the claims.

### BRIEF DESCRIPTION OF THE DRAWINGS

The accompanying drawings, which are included to provide a further understanding of the disclosed subject matter, are incorporated in and constitute a part of this specification. The drawings also illustrate embodiments of the disclosed subject matter and together with the detailed description serve to explain the principles of embodiments of the disclosed subject matter. No attempt is made to show structural details in more detail than may be necessary for a fundamental understanding of the disclosed subject matter and various ways in which it may be practiced.

FIG. 1(a) shows a Ti—H phase diagram and FIG. 1(b) shows a Ti equilibrium phase diagram according to the disclosed subject matter.

FIG. 2 shows a schematic diagram of a fixed-bed aromatization reactor with catalyst and titanium alloy hydrogen acceptor particles intermixed in a fixed bed or stationary configuration according to an embodiment of the disclosed subject matter.

FIG. 3 shows a schematic diagram of regeneration of the intermixed catalyst and titanium alloy hydrogen acceptor particles in a single regeneration vessel according to an embodiment of the disclosed subject matter.

FIG. 4 shows a schematic diagram of separation and regeneration of catalyst and titanium alloy hydrogen acceptor particles in separate vessels according to an embodiment of the disclosed subject matter.

FIG. 5 shows example structural variations generated for Ti—V alloy compositions according to various embodiments of the disclosed subject matter.

FIG. 6 shows the relationship between temperature and relative phase stability of Ti—V alloys based on various embodiments of the disclosed subject matter.

FIG. 7(a), (b), (c) show composition of alloying element vanadium in single-phase titanium alloy stabilized in beta-phase across various temperatures according to an embodiment of the disclosed subject matter.

FIGS. 8(a) and (b) show structural variants of an example titanium alloy in (a) beta and (b) alpha phase structures according to an embodiment of the disclosed subject matter.

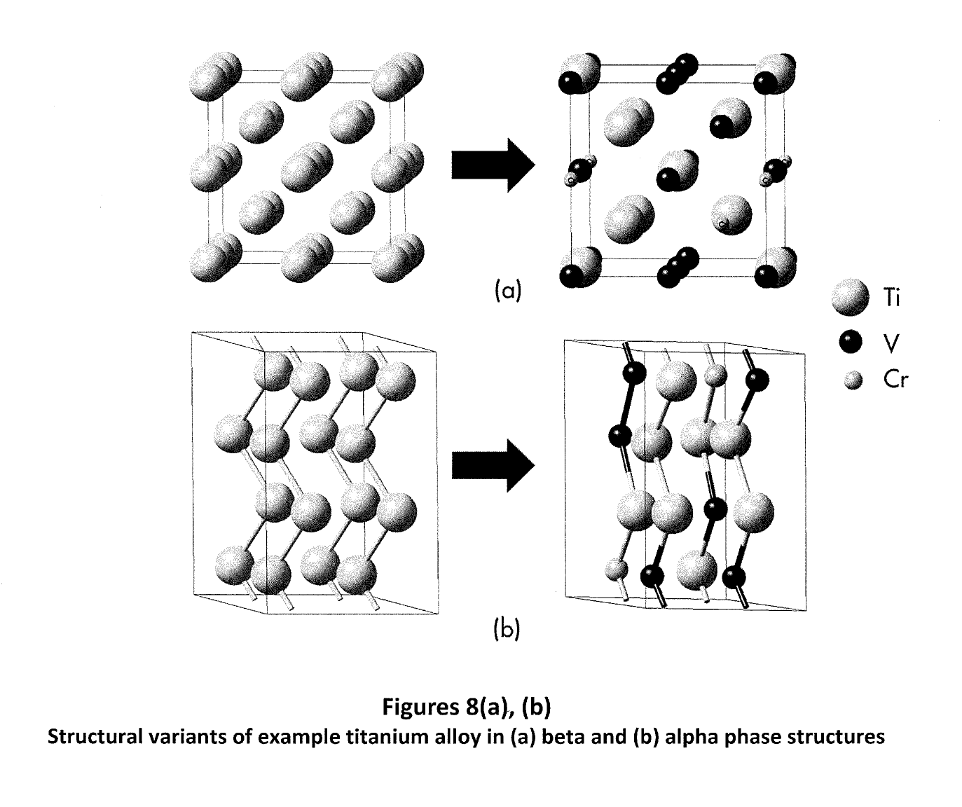

### DETAILED DESCRIPTION

The conversion of a methane-containing gas stream to aromatics is typically carried out in a reactor comprising a solid catalyst substance, which is active in the conversion of the methane-containing gas stream to aromatics. The methane-containing gas stream that is fed to the reactor comprises more than 50% vol. methane, preferably more than 70% vol. methane and more preferably of from 75% vol. to 100% vol. methane. The balance of the methane-containing gas may be other low molecular weight alkanes, for example, ethane, propane and butane. The methane-containing gas stream may be natural gas which is a naturally occurring hydrocarbon gas mixture consisting primarily of methane, with up to about 30% vol. concentration of other hydrocarbons (usually mainly ethane and propane), as well as small amounts of other impurities such as carbon dioxide, nitrogen and others. The aromatization reaction of this invention is carried out in a reactor, for example, a fixed bed reactor. To enable this, suitably shaped and sufficiently robust catalyst and titanium alloy hydrogen acceptor particles that are able to sustain the rigors of high severity reactor operation are prepared and used for the reaction.

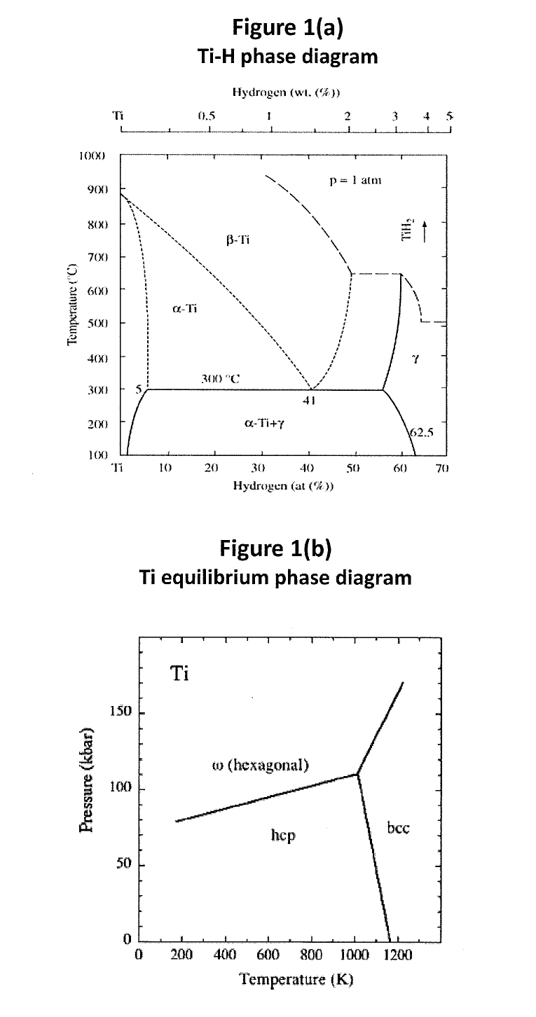

In general, the use of specific operating conditions for the aromatization process and hydrogen acceptors in the reaction zone provides several advantages. Hydrogen acceptors shift the thermodynamic equilibrium of the methane-to-benzene (M2B) conversion reaction. In particular, zirconium (Zr) and titanium (Ti) metals are examples of hydrogen acceptors that may be employed under known M2B reaction conditions. For example, by physically mixing Zr metal and catalyst particles under M2B reaction conditions does indeed result in a dramatic shift in the thermodynamic equilibrium of the M2B reaction in favor of the desired benzene product, as demonstrated in related co-pending patent applications WO2013/1631116 A1 and WO2013/163118 A1, which are incorporated by reference herein. However, inspection of the Ti—H phase diagram (see FIG. 1(a)) reveals that Ti undergoes multiple phase transitions during hydrogen uptake as a function of increasing hydrogen concentration. This may have implications in volumetric changes of Ti—H solid solutions and the corresponding stoichiometric hydrides. The multiple hydrogen solid solutions and associated volumetric changes have the following deleterious impact on M2B reactions:

(1) Repeated expansion/contraction may result in the generation of significant stresses within the hydrogen acceptor particle causing cracking. This threat to mechanical integrity of these particles may be disruptive for mechanical handling of these particles towards the scheme of the M2B reaction via continuous H-adsorption and regeneration of H₂-acceptor materials.

(2) Added complexity arises due to varying rates of heat transfer and H-diffusion through multiple hydride phases, which may impact the hysteresis of adsorption/desorption over multiple cycles of adsorption and regeneration.

Therefore, given this need for cyclic stability (both in terms of chemical potential and mechanical integrity) of the H₂-acceptor material, there is a need for an improved H₂ acceptor material that accepts hydrogen and maintains phase stability throughout the operation at the desired temperatures and pressure in the M2B process.

The present invention provides for high-temperature hydrogen acceptor material in form of a single-phase titanium alloy, suitable for use in the direct, non-oxidative, heterogeneous catalytic methane aromatization reaction.

In particular, the titanium alloy H₂ acceptor reversibly binds the hydrogen in such a way that during operation in the reactor, the hydrogen is strongly bound to the titanium alloy H₂ acceptor under the methane containing gas aromatization conditions. In addition, the titanium alloy H₂ acceptor is able to release the hydrogen when subjected to regeneration conditions that favor release of the previously bound hydrogen and regeneration of the titanium alloy H₂ acceptor. The present invention provides an efficient, high temperature titanium H₂ acceptor material that is capable of shifting the thermodynamic equilibrium of the methane aromatization reaction to achieve significantly higher CH₄ conversion and benzene yields than the maximum allowable without use of the titanium alloy H₂ acceptor.

The present invention provides a Ti alloy (as opposed to pure Ti) hydrogen acceptor which preserves a single phase structure of the hydrogen acceptor, even upon hydriding. The presently disclosed alloy is made of titanium, in conjunction with one or more other metals such as Zr, Hf, V, Nb, Ta, Mo, Re, Cr, Mn, Fe, Co, Ni, Cu, Pd, Pt, Ag, Au, and W. This Ti alloy adsorbs hydrogen as solid solution phase in large amounts, serving the basic purpose of a hydrogen acceptor material, while staying as a single-phase structure thereby improving the cyclic stability in operation. Accordingly, the present invention significantly improves the methane to aromatics reaction efficiency and the potential for commercialization of a methane aromatization process.

The usage of titanium alloy hydrogen acceptor particles in a reactor when operating under aromatization conditions provides for the quick removal of the produced hydrogen from the reaction zone and for shifting the aromatization reaction equilibrium toward greater methane conversion and benzene yield per pass. The titanium alloy hydrogen acceptor used in this reaction, when subjected to aromatization operating conditions, selectively accepts, absorbs or reacts with hydrogen to form a sufficiently strong titanium-hydrogen bond (such as for example in titanium stoichiometric hydride or solid solution titanium hydride). The titanium alloy hydrogen acceptor reversibly binds the hydrogen in such a way that during operation in the reactor the hydrogen is strongly bound to the titanium alloy acceptor under the methane-containing gas stream aromatization conditions. In addition, the titanium alloy hydrogen acceptor is preferably able to release the hydrogen when transported to the regeneration section where it is subjected to a different set of (regeneration) conditions that favor release of the previously bound hydrogen and regeneration of the titanium alloy hydrogen acceptor.

A feature of the methane aromatization process according to the present invention is the use of single-phase Ti alloys as hydrogen acceptor materials, which maintains the same phase structure even after H-adsorption, thus minimizing phase-change stresses avoiding decrepitation during M2B reaction cycling and subsequent regeneration. The stable phase for pure Ti under atmospheric pressure conditions is HCP (Hexagonal-Close-Packed, also known as α), which transforms to BCC (Body-Centered-Cubic, also known as β) around 882° C., as shown in the phase diagram in FIG. 1(b).

Alloying elements added to pure Ti may stabilize one phase over the other. Even though there are equal opportunities of stabilizing alpha-phase of Ti using other alloying elements, the hydrogen acceptor material according to the present invention is specifically designed to be stable in the beta-phase in Ti for the following reasons:
(1) Solubility of hydrogen in β-phase is significantly greater than the solubility of hydrogen in α-phase. Hence from a hydrogen acceptance perspective, a beta-phase Ti alloy may have a higher H-adsorption capacity and hence more suited than alpha-Ti-alloy for boosting the M2B reaction conversion
(2) Hydrogen in β-phase adds stability to the β-phase; i.e., hydrogen is a β-stabilizer. Hydrogen is found to lower the β-transus by 10-15° C. per atomic %. Hence beta-phase Ti alloy compositions are more likely to maintain their phase stability upon hydrogen adsorption.
(3) Also, diffusivity of hydrogen in β-phase is much faster than diffusivity of hydrogen in α-phase. This may boost the M2B kinetics in the reactor by promoting faster removal of H₂ from the gas stream, as well as faster regeneration of the hydrogen acceptor for its reuse.

Accordingly, elements that widen the window of β-stability and are found to promote lower transformation temperatures, known as β-stabilizers. The presently disclosed alloy is made of titanium, in conjunction with one or more other metals such as Zr, Hf, V, Nb, Ta, Mo, Re, Cr, Mn, Fe, Co, Ni, Cu, Pd, Pt, Ag, Au, and W, which when added to pure Ti in different characteristic amounts, increase β-stability.

The shaped titanium alloy hydrogen acceptor particles may be in the form of irregular particles, cylindrical pellets, rings, tablets or spheres. The preferred titanium alloy hydrogen acceptor particle shapes are pellets, rings or spheres. The preferred particle size of the titanium alloy hydrogen acceptor of this invention is preferably selected to be in the range of 50 micron to 2 mm.

The conversion of a methane-containing gas stream is carried out at particular operating conditions which lead to improved conversion and benzene yields. For example, the process of the present invention may be carried out at a gas hourly space velocity of from 100 to 40,000 h⁻¹, a pressure of from 0.5 to 10 bara and a temperature of from 500 to 900° C. More preferably, the conversion is carried out at a gas hourly space velocity of from 300 to 30,000 h⁻¹, a pressure of from 0.5 to 5 bara and a temperature of from 600 to 800° C. Even more preferably, the conversion is carried out at a gas hourly space velocity of from 500 to 10,000 h⁻¹, a pressure of from 0.5 to 3 bara and a temperature of from 650 to 750° C. In an embodiment, the pressure may be at least 2 bara, and according to an embodiment, the pressure may be at least 3 bara. The methane aromatization reaction is carried out until conversion falls to values that are lower than those that are economically acceptable. At this point, the aromatization catalyst has to be regenerated to restore its aromatization activity to a level similar to its original activity. The regeneration of the catalyst could be carried out separately from the titanium alloy H₂ acceptor or in the presence of the titanium alloy H₂ acceptor. Also the regeneration of the titanium alloy H₂ acceptor could be carried out separately from the catalyst or in the presence of the catalyst. Following the regeneration, the catalyst is again contacted with the titanium alloy H₂ acceptor and a methane-containing gas stream in the reaction zone of the aromatization reactor for continuous production of aromatics.

Any catalyst suitable for methane-containing gas aromatization can be used in the process of this invention. The catalyst typically comprises one or more active metals on an inorganic oxide support and optionally comprises promoters and other beneficial compounds. The active metal or metals, promoters, compounds and the inorganic support all contribute to the overall aromatization activity, mechanical strength and performance of the aromatization catalyst.

The active metal component(s) of the catalyst may be any metal that exhibits catalytic activity when contacted with a methane-containing gas stream under methane aromatization conditions. The active metal may be selected from the group consisting of: vanadium, chromium, manganese, zinc, iron, cobalt, nickel, copper, gallium, germanium, niobium, molybdenum, ruthenium, rhodium, silver, tantalum, tungsten, rhenium, platinum and lead and mixtures thereof. The active metal is preferably molybdenum.

The promoter or promoters may be any element or elements that, when added in a certain preferred amount and by a certain preferred method during catalyst synthesis, improve the performance of the catalyst in the methane aromatization reaction.

The inorganic oxide support can be any support that, when combined with the active metal or metals and optionally the promoter or promoters contributes to the overall catalyst performance exhibited in the methane aromatization reaction. The support has to be suitable for treating or impregnating with the active metal compound or solution thereof and a promoter compound or solution thereof. The inorganic support preferably has a well-developed porous structure with a sufficiently high surface area and pore volume and suitable for aromatization surface acidity. The inorganic oxide support may be selected from the group consisting of zeolites, non-zeolitic molecular sieves, silica, alumina, zirconia, titania, yttria, ceria, rare earth metal oxides and mixtures thereof. The inorganic oxide support of this invention preferably contains zeolite as the primary component. The zeolite is selected from the group consisting of ZSM-5, ZSM-22, ZSM-8, ZSM-11, ZSM-12 or ZSM-35 zeolite structure types. The zeolite is preferably a ZSM-5 zeolite. The ZSM-5 zeolite further may have a SiO₂/Al₂O₃ ratio of 10 to 100. Preferably, the SiO₂/Al₂O₃ ratio of the zeolite is in the range of 20-50. Even more preferably the SiO₂/Al₂O₃ ratio is from 20 to 40 and most preferably from about 20 to 30. The support may optionally contain about 15-70% wt of a binder that binds the zeolite powder particles together and allows for shaping of the catalyst in the desired form and for achieving the desired high catalyst mechanical strength. More preferably the support contains from 15-30% wt. binder. The binder is selected from the group consisting of silica, alumina, zirconia, titania, yttria, ceria, lanthana, and other rare earth oxides or mixtures thereof.

The final shaped catalyst could be in the form of cylindrical pellets, rings or spheres. The preferred catalyst shape of this invention is spherical or cylindrical pellets. The spherical or pelletized catalyst of this invention could be prepared by any method known to those skilled in the art. Preferably, the spherical catalyst of this invention is prepared via spray drying of zeolite containing sols of appropriate concentration and composition. The zeolite containing sol may optionally contain binder. The spherical catalyst has a particle size distribution and predominant particle size or diameter that makes it suitable for use in the disclosed process. The spherical particle diameter of the catalyst of this invention is preferably selected to be in the range of 20 microns to 3 mm. More preferably, the spherical catalyst of this invention has particle diameter in the range of 50 microns to 2 mm. As an example, particle size may be based on the prevalent particle size measured from a particle size distribution. For example, if a particle size distribution is measured (e.g., using the light scattering method) of the spherical catalyst particles, the prevalent particle size may appear as a peak in a plot of the number of particles versus particle size. The cylindrical pelletized catalyst of this invention is prepared by extrusion of suitable extrusion mix containing appropriate concentration of zeolite powder and optionally binder. The diameter of the cylindrical catalyst pellets is selected to be in the range of from 1 to 4 mm.

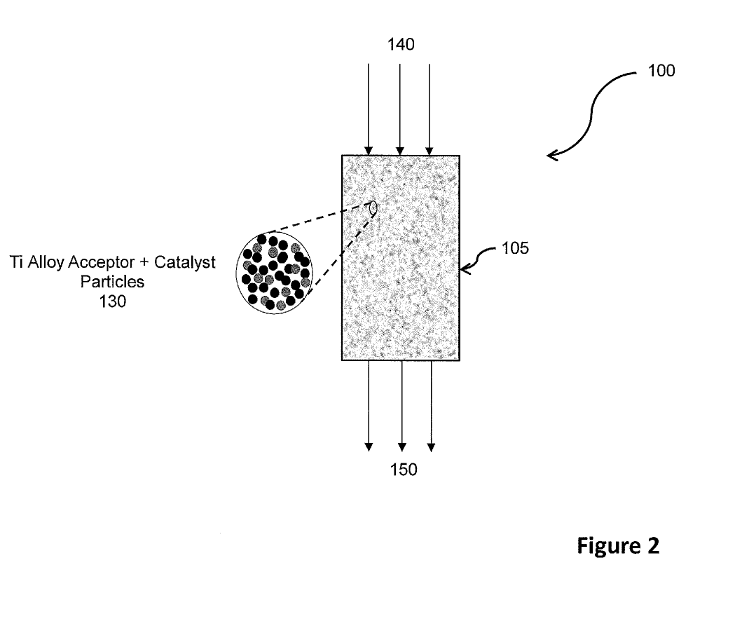

Another feature of the methane aromatization process of this invention is that it provides for insitu removal of hydrogen from the reaction zone and significant thermodynamic equilibrium shift by use of a titanium alloy hydrogen acceptor particles combined with catalyst particles in the reaction zone. As a result, a significant advantage of the disclosed subject matter is that it provides for a substantial increase in both methane conversion and benzene yield per pass. This results in methane conversion and benzene yield values that are significantly higher relative to those achieved by the same methane aromatization reaction but without use of the presently disclosed titanium alloy hydrogen acceptor. As an example, FIG. 2 shows a fixed-bed aromatization reactor with catalyst and titanium alloy hydrogen acceptor particles intermixed in a fixed bed configuration. As shown, a reactor 100 with a fixed bed 105 comprises a mixture 130 of catalyst and titanium alloy hydrogen acceptor particles. The process gas may flow downward into the fixed bed 105 through gas inlet 140 and outward from the fixed bed 105 through gas outlet 150, as shown by the arrows 140 and 150.

Another advantage of the present invention is that, the particle shapes, sizes and mass of both titanium alloy hydrogen acceptor and catalyst particles can be designed and selected in such a way so that they can be combined and mixed well together in the reactor volume. In addition, they could be designed in such a way so that to provide for easy separation of particles by type following the reaction and prior to regeneration in separate vessels.

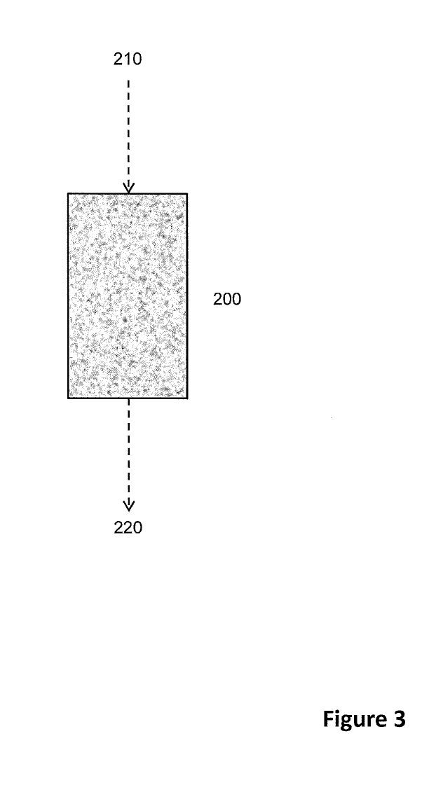

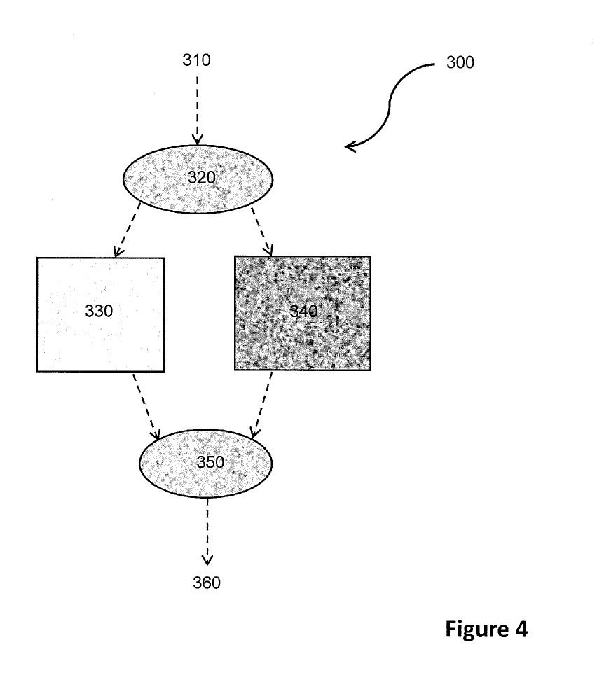

Another advantage of the process of this invention is that it provides for the catalyst and the titanium alloy hydrogen acceptor particles to be simultaneously regenerated in the reactor (e.g., as shown in FIG. 2) or withdrawn from the reaction zone, regenerated in a separate vessel or vessels according to one of the schemes illustrated in FIGS. 3 and 4 and then returned to the reactor for aromatics and hydrogen production. In addition, a method of regenerating the titanium alloy hydrogen acceptor and reusing it in the M2B reaction to afford performance very similar to the one of the fresh titanium acceptor is also provided. The titanium alloy hydrogen acceptor and catalyst regeneration can be accomplished either simultaneously or stepwise in the reactor illustrated in FIG. 2 or in a different regeneration vessel as illustrated in FIG. 3 or regenerated separately in separate vessels as illustrated in FIG. 4. The later operation schemes (e.g., as shown in FIGS. 3 and 4) provide for maximum flexibility to accomplish the hydrogen release or regeneration of the titanium alloy acceptor and catalyst particles under different and suitable for the purpose operating conditions. The regeneration of the catalyst and titanium alloy hydrogen acceptor can be accomplished in fixed, moving or fluidized bed reactor vessels schematically shown in FIGS. 2-4. In the specific case of separate regeneration as illustrated in FIG. 4, the titanium alloy hydrogen acceptor particles can be separated from the catalyst on the basis of (but not limited to) differences in mass, particle size or density between the titanium alloy acceptor and the catalyst particles.

FIG. 3 shows a regenerator vessel 200 that is used to regenerate the catalyst and regenerate the titanium alloy hydrogen acceptor. The catalyst and titanium alloy hydrogen acceptor particles are introduced via inlet 210 and are then removed following the regeneration via outlet 220. During the regeneration, the regeneration gas could be fed downward in the direction from 210 to 220 or upward in the direction from 220 to 210. The hydrogen removed from the titanium alloy hydrogen acceptor during the regeneration and the gases produced during catalyst regeneration are removed from the regenerator via 210 or 220 or if needed, additional outlets (not shown).

In FIG. 4, regenerator system 300 comprises a separation step 320 to separate the catalyst from the titanium alloy hydrogen acceptor. First, a mixture of spent catalyst and titanium acceptor particles are fed from the reactor via line 310. Following the separation in 320, the catalyst particles are fed to the catalyst regeneration vessel 330, and the titanium alloy hydrogen acceptor particles are fed to titanium alloy hydrogen acceptor regeneration vessel 340. The regenerated catalyst and titanium alloy hydrogen acceptor particles are then mixed back together in mixing step 350 and then fed back to the reactor via line 360.

The methane aromatization catalyst forms coke during the reaction. An accumulation of coke on the surface of the catalyst gradually covers the active aromatization sites of the catalyst resulting in gradual reduction of its aromatization activity. Therefore, the coked catalyst has to be regenerated at a certain carefully chosen frequency insitu in the reactor as illustrated in FIG. 2 or removed from the reaction zone of the aromatization reactor and regenerated in one of the regeneration vessel(s) as illustrated in FIGS. 3 and 4. The regeneration of the catalyst could be carried out by any of the methods known to those skilled in the art while the titanium alloy hydrogen acceptor particles are completely withdrawn or still within the reaction zone of the reactor.

The regeneration of the catalyst can be carried out by any method known to those skilled in the art. For example, two possible regeneration methods are hot hydrogen stripping and oxidative burn at temperatures sufficient to remove the coke from the surface of the catalyst. If hot hydrogen stripping is used to regenerate the catalyst, then at least a portion of the hydrogen used for the catalyst regeneration may come from the hydrogen released from the titanium alloy hydrogen acceptor. Additionally, fresh hydrogen may be fed to the catalyst regeneration vessel as needed to properly supplement the hydrogen released from titanium alloy hydrogen acceptor and to complete the catalyst regeneration. If the catalyst and titanium acceptor regeneration is carried out in the same vessel (see FIGS. 2-3), then the hydrogen removed from the titanium alloy hydrogen acceptor insitu or exsitu can at least partially hydrogen strip and regenerate the catalyst.

If the regeneration of catalyst and titanium alloy hydrogen acceptor particles is carried out in different vessels, the operating conditions of each vessel can be optimized, selected and maintained to favor the regeneration of the catalyst or the titanium alloy hydrogen acceptor, respectively. Hydrogen removed from the titanium alloy hydrogen acceptor can be used to at least partially hydrogen strip and regenerate the catalyst.

Yet another advantage of the process of this invention is that it provides for the release of the hydrogen that is bound to the titanium alloy hydrogen acceptor when the saturated acceptor is subjected to the regeneration conditions in the regeneration vessel(s). Furthermore, the released hydrogen can be utilized to regenerate the catalyst, or it may be subjected to any other suitable chemical use, or monetized to improve the overall aromatization process economics.

Another advantage of the present invention is that it allows for different regeneration conditions to be used in the different regeneration vessels to optimize and minimize the regeneration time required for the catalyst and titanium alloy hydrogen acceptor and to improve their performance in the methane aromatization reaction.

### EXAMPLES

Various tests and calculations were performed using first-principles Density Functional Theory (DFT) (see e.g., P. Hohenberg, W. Kohn, Phys. Rev. B136 (1964) 864; W. Kohn, L. J. Sham, Phys. Rev. 140 (1965) A1133), combined with Phonon (see e.g., K. Parlinski, Z. Q. Li, and Y. Kawazoe, Phys. Rev. Lett. 78 (1997) 4063) calculations to demonstrate various alloying additions in Ti that result in a single-phase Ti alloy that is stable in β phase.

Method:

To determine the effect of the concentration and atomic distribution of the alloying elements on the phase stability, ordered structure models and special quasirandom structure (SQS) models were used for emulating the solid solution state of the alloys.

To perform density functional theory calculations, the projected augmented wave method (see e.g., P. E. Blöchl, Projector augmented-wave method, Phys. Rev. B 50 (1994) 17953; G. Kresse, D. Joubert, Phys. Rev. B 59 (1999) 1758) was used as implemented in the Vienna Ab initio simulation package (see e.g., G. Kresse, J. Hafner, Phys. Rev. B 47 (1993) 558; G. Kresse, J. Furthmüller, Phys. Rev. B 54 (1996) 11169). The exchange-correlation energy was calculated within the generalized gradient approximation (GGA) proposed by Perdew and Wang (see e.g., J. P. Perdew, Y. Wang, Phys. Rev. B 45 (1992) 13244). The total energy was minimized over the degrees of freedom of the electron density and the ionic positions and structural parameters. Electronic convergence was set as 10⁻⁴ eV per atom for all cases. The cut-off energy for the plane wave expansion was set at 400 eV. Brillouin zone integrations were performed using a set of 4×4×4 k-points.

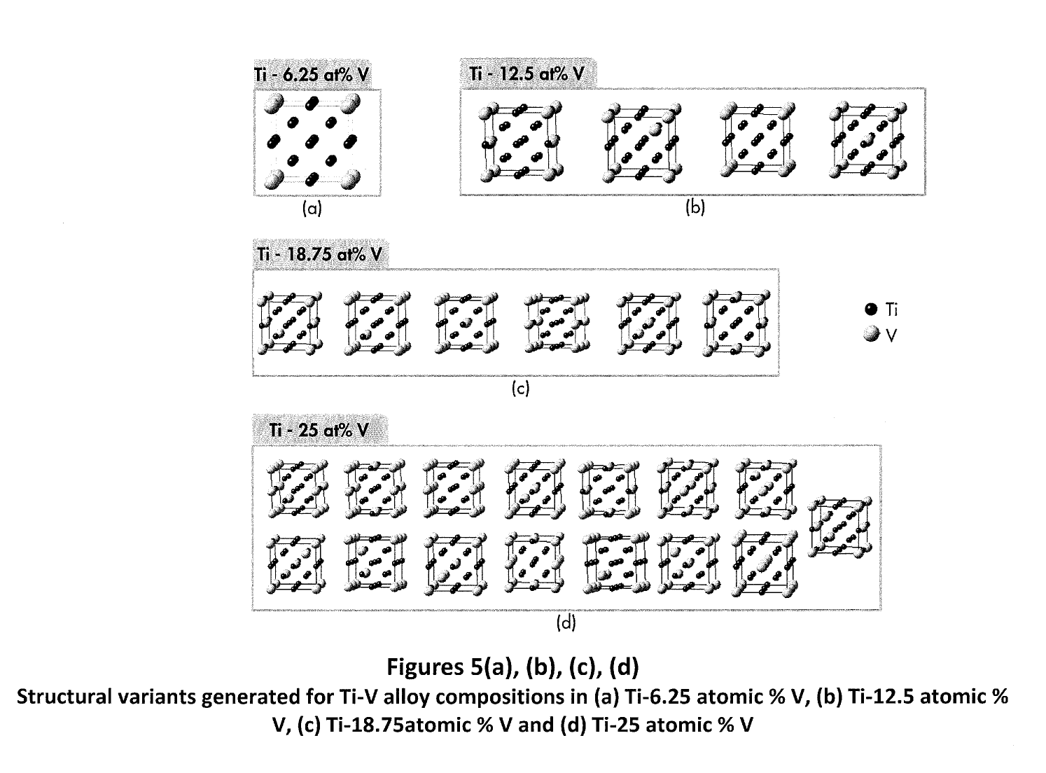

The nonstoichiometric concentrations of Ti—X alloys were described with the 16-atom systems constructed as a 2×2×2 supercell in both BCC and HCP crystal structures. SQS models were generated for eight nonstoichiometric concentrations: Ti0:9375X0:0625, Ti0:8750X0:1250, Ti0:8125X0:1875, Ti0:750X0:250, Ti0:6875X0:3125, Ti0:6250X0:3750, Ti0:5625X0:4375, and Ti0:500X0:500 as shown in FIG. 5. A total of 740 SQS models were generated as shown below in Table 1.

TABLE 1
Total number of structural variants generated in BCC and HCP structures
of Ti—V alloys, as a function of alloy compositions.

| | Atomic % V | Structural Variants | Structural Variants |
|---|---|---|---|
| Composition | in Ti | in BCC Ti | in HCP Ti |
| Ti15V | 6% | 1 | 1 |
| Ti14V2 | 13% | 5 | 5 |
| Ti13V3 | 19% | 6 | — |
| Ti12V4 | 25% | 17 | 30 |
| Ti11V5 | 31% | 18 | 49 |
| Ti10V6 | 38% | 29 | 86 |
| Ti9V7 | 44% | 31 | 99 |
| Ti8V8 | 50% | 60 | 190 |
| Ti7V9 | 56% | 33 | |
| Ti6V10 | 63% | 30 | |
| Ti5V11 | 69% | 18 | |
| Ti4V12 | 75% | 20 | |
| Ti3V13 | 81% | 6 | |
| Ti2V14 | 88% | 5 | |
| TiV15 | 94% | 1 | |

| | |
|---|---|
| TOTAL | 740 |

To analyze the relative phase stabilities of Ti—V alloys, formation energies of these alloys were calculated as:

E_{f}(TiₓV_{y})=E(TiₓV_{y})−x*E(Ti)−y*E(V), where

E(TiₓV_{y}) is the total energy of a TiₓV_{y} alloy with x Ti and y V atoms;
E(Ti) and E(V) are the energies of Ti and X in their ground state phases.
The formation energies of the Ti—V alloys as estimated correspond to their formation energies at 0 K, without entropic contributions.

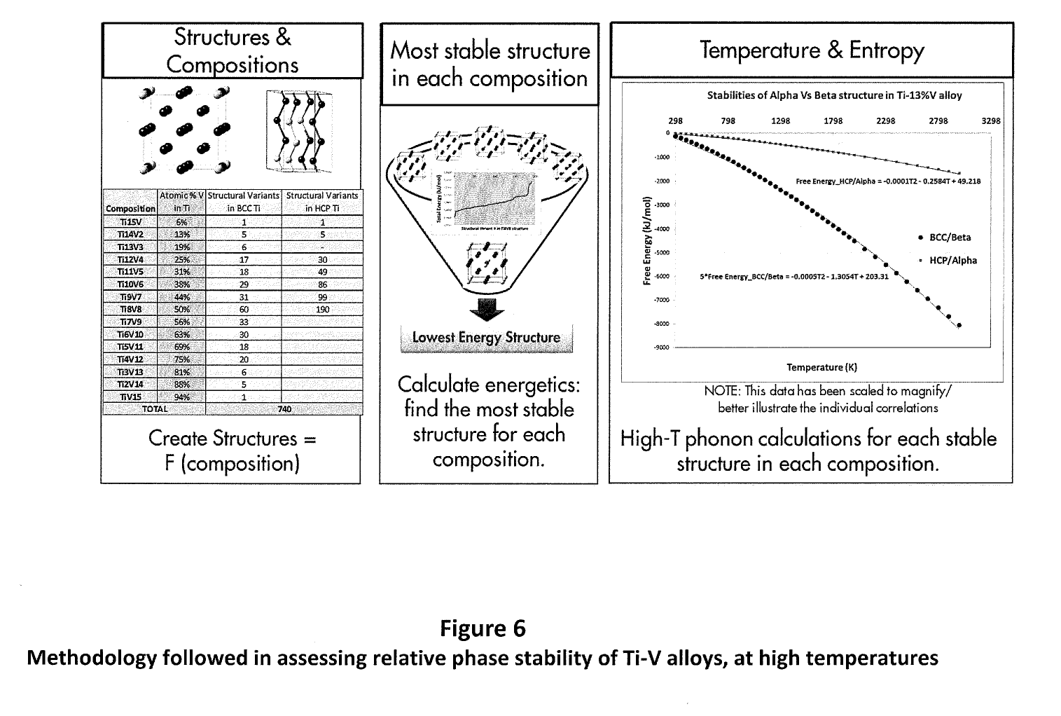

To estimate the formation energies of Ti—V alloys in the preferred temperature range within M2B reaction operating conditions, phonon calculations were performed based on the supercell method (e.g., see K. Parlinski, Z. Q. Li, and Y. Kawazoe, Phys. Rev. Lett. 78 (1997) 4063), starting from the lowest energy (most stable) structure from each composition. Force constants, i.e. the Hessian matrix, were calculated in real space using a 2×2×2 supercell containing (70-150) atoms, depending on the different Ti—V phases and their configurations. The large number of symmetry-unique atomic sites required (70-120) VASP single point energy calculations for the different Zr—H phases. Atomic displacements of ±0.02 Å were employed for the independent atoms and an average k-spacing of 0.25 Å⁻¹ was used for each single point energy calculation. FIG. 6 shows the methodology followed in assessing relative phase stability of Ti—V alloys at high temperatures according to an embodiment of the present invention.

Results:

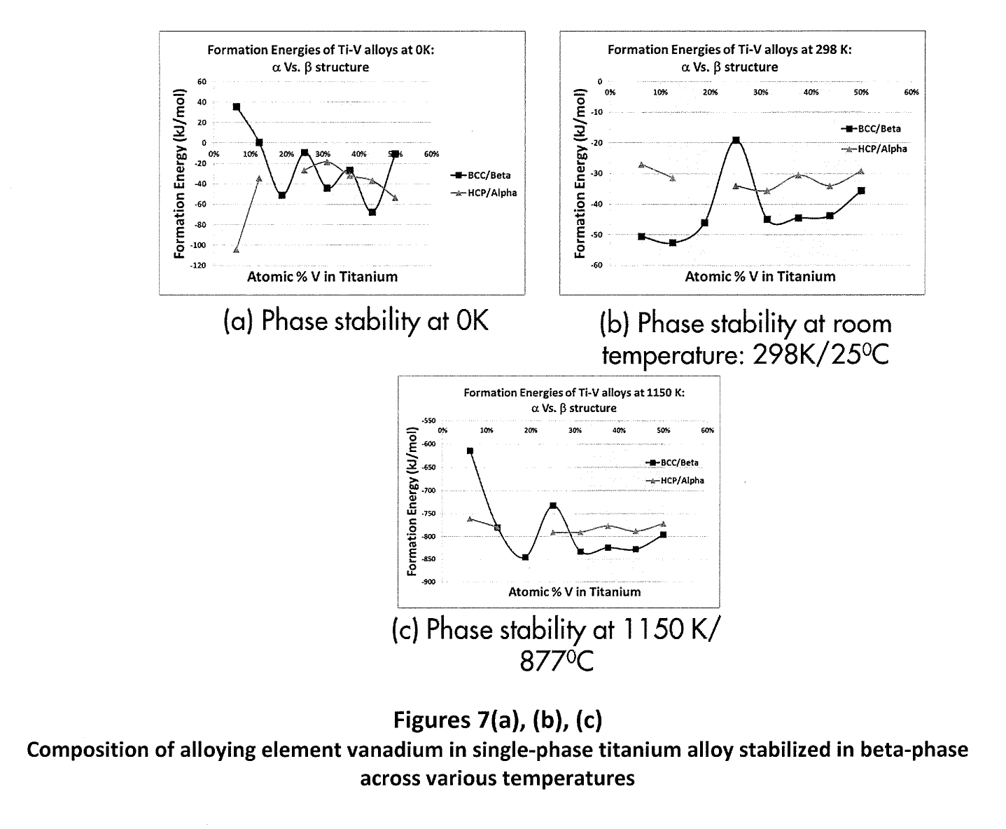

As demonstrated in the calculations performed, the composition of alloying element vanadium in titanium necessary to stabilize the single-phase titanium alloy in beta-phase across the preferred temperature of operation, e.g., 0 K to greater than 850° C. is shown in FIG. 7(a), (b), and (c). In particular, FIG. 7(a) shows that at 0 K and without entropic effects 31-44 atomic % V in Ti stabilizes the beta phase over the alpha phase. As shown in FIG. 7(b), at 298 K, 6-13 atomic % V and 31-50 atomic % V in Ti stabilizes the single-phase titanium alloy in beta-phase. However, as shown in FIG. 7(c) at higher temperatures such as 1150 K/877 C, 31-50 atomic % V in Ti is needed to stabilize the beta phase.

Hence, these calculations demonstrate that within the preferred temperature range of operation for the M2B process from room temperature to greater than 800° C., Ti and 31-50 atomic % V is suitable for keeping the titanium alloy in a single-phase, specifically beta phase.

As another example of the presently disclosed subject matter, a commercial ternary titanium alloy was tested for phase stability. The composition of this titanium alloy is: Ti, 35 weight % V, 15 weight % Cr. Thermodynamic stability of alpha and beta phases in this alloy were calculated following same procedure described above and shown in FIG. 6.

The calculations demonstrate that the beta phase in the titanium alloy comprising vanadium and chromium is more stable than the alpha phase by 15 kJ/mol. See Table 2 below.

TABLE 2

| | Total Energy (kJ/mol) for Ti₄V₃Cr |
|---|---|
| Beta-Phase | −6486.156 |
| Alpha-Phase | −6471.610 |

As shown, the addition of 35 wt % V and 15 wt % Cr in Ti stabilizes the alloy in a single-phase, and particularly in beta phase according to the disclosed subject matter.

Based on the above calculations, other alloying elements which may behave similar to V in terms of promoting single phase beta in Ti and its solid solution hydride would be other transition metals having similar atomic sizes and electronegativity as V, such as Cr, Mo, Nb, and others.

Other Ti-alloy compositions are:
(1) Binary Ti—V alloys with compositions ranging from (30-50) atomic % V in Ti
(2) Binary Ti—Mo alloys with compositions ranging from (30-50) atomic % Mo in Ti
(3) Ternary Ti—Cr—V alloys with compositions ranging from equiatomic Ti—Cr—V, i.e. Ti33Cr33V33 to increasing proportions of Cr in the alloy up to 60 atomic %, balance being Mo or V; for examples compositions Ti33Cr60Mo7, Ti33Cr60V7
(4) Ternary Ti—V—Cr alloys with compositions ranging from 30-50 atomic % V, 10-20 atomic % Cr, balance Ti. For example, commercially available alloy with composition Ti, 35 weight % V, and 15 weight % Cr (translates to Ti, 34.02 atomic % V, and 14.28 atomic % Cr).

The data presented above clearly shows that the single-phase alloys based on a single-phase titanium alloy as hydrogen acceptor according to the disclosed subject matter alleviates the issues with phase stability, mechanical integrity and degradation over M2B reaction cycling. Therefore, this invention provides for overcoming of the most significant challenge on the path of commercializing an efficient and economically attractive methane aromatization process.

The foregoing description, for purpose of explanation, has been described with reference to specific embodiments. However, the illustrative discussions above are not intended to be exhaustive or to limit embodiments of the disclosed subject matter to the precise forms disclosed. Many modifications and variations are possible in view of the above teachings. The embodiments were chosen and described in order to explain the principles of embodiments of the disclosed subject matter and their practical applications, to thereby enable others skilled in the art to utilize those embodiments as well as various embodiments with various modifications as may be suited to the particular use contemplated.

What is claimed is:

1. A process for the aromatization of a methane-containing gas stream comprising:
contacting the methane-containing gas stream in a reaction zone of an aromatization reactor comprising an aromatization catalyst and a titanium alloy hydrogen acceptor under methane-containing gas aromatization conditions to produce a product stream comprising aromatics and hydrogen, wherein at least a portion of the produced hydrogen is bound by the titanium alloy hydrogen acceptor in the reaction zone and removed from the product stream and the reaction zone, and
wherein the titanium alloy hydrogen acceptor is a single phase alloy.

2. The process of claim 1, wherein the titanium alloy hydrogen acceptor is in beta phase.

3. The process of claim 1, wherein the methane-containing gas stream conversion and corresponding benzene yield per pass are higher than the conversion and yield obtained with the same aromatization catalyst and under the same methane-containing gas aromatization conditions, but in the absence of the titanium alloy hydrogen acceptor in the reaction zone of the aromatization reactor.

4. The process of claim 1, wherein the titanium alloy hydrogen acceptor comprises one or more metals selected from the group consisting of: Zr, Hf, V, Nb, Ta, Mo, Re, Cr, Mn, Fe, Co, Ni, Cu, Pd, Pt, Ag, Au, W.

5. The process of claim 1, wherein the titanium alloy hydrogen acceptor comprises vanadium.

6. The process of claim 1, wherein the titanium alloy hydrogen acceptor comprises molybdenum.

7. The process of claim 1, wherein the titanium alloy hydrogen acceptor comprises chromium and vanadium.

8. The process of claim 1, wherein the obtained conversion of the methane-containing gas stream is at least 35 wt %.

9. The process of claim 1, wherein the obtained benzene yield per pass is at least 15 wt %.

10. The process of claim 1, wherein the methane-containing gas stream further comprises at least one compound selected from the group consisting of ethane, propane, butane, and carbon dioxide.

11. The process of claim 1, wherein the aromatization reactor is a fixed bed reactor.

12. The process of claim 1, wherein the methane aromatization conditions comprise a temperature in the range of from 500° C. to 900° C.

13. The process of claim 1, wherein the methane aromatization conditions comprise a temperature in the range of from 600° C. to 800° C.

14. The process of claim 1, further comprising continuously regenerating the catalyst to remove coke formed during the reaction and continuously regenerating the titanium alloy hydrogen acceptor by releasing the hydrogen under regeneration conditions.

15. The process of claim 14, wherein the catalyst and hydrogen acceptor are regenerated in separate vessels.

16. The process of claim 1, wherein the catalyst and hydrogen acceptor are each regenerated under different regeneration conditions.

17. The process of claim 14, wherein the hydrogen released from the hydrogen acceptor during regeneration of the hydrogen acceptor is used for catalyst regeneration.

18. The process of claim 17, wherein supplemental hydrogen is supplied from an external source in order to properly complete the catalyst regeneration.

19. The process of claim 14, wherein the titanium hydrogen acceptor regeneration is accomplished under regeneration conditions including: feed rate, temperature and pressure that are substantially different from the aromatization conditions.

20. The process of claim 14, wherein the titanium acceptor regeneration conditions include a regeneration gas GHSV of from 500-10,000 h-1, a temperature of from 700-950° C. and pressure of from 0.5-4 bara.
